(19) United States
(12) Patent Application Publication
(10) Pub. No.: US 2005/0265942 A1
(43) Pub. Date: Dec. 1, 2005

(54) Compositions using evaporable silicone carriers for cosmetics, cleaning and care product compositions

(76) Inventors: Suresh K. Rajaraman, Newburgh, NY (US); Mark D. Leatherman, Elmsford, NY (US); Roy U. Rojas-Wahl, Teaneck, NJ (US); John S. Razzano, Cohoes, NY (US)

Correspondence Address: Kenneth S. Wheelock;General Electric Company, One Plastics Avenue, Pittsfield, MA, 01201, US

(21) Appl. No.: 10/854,037

(22) Filed: May 26, 2004

(51) Int. Cl. A61K 7/11

(52) U.S. Cl. 424/70.12

(57) ABSTRACT

This invention describes cosmetic, cleaning and care products compositions made using an evaporable siloxane liquid support or carrier having the molecular formula:
MᵥM′_{w}DₓT_{y}Q_{z} where the subscripts may have the following values: x=0, 1, 2, 3, or 4; y=0, 1, or 2; z=0, 1, 2, 3, or 4; and v and w may be zero or a positive integer subject to the limitation that v+w≧1 where the components are defined as follows:
M=(R¹O)ₐ(R²O)_{b}R³_{c}SiO_{1/2} where the subscripts may have the following values: a=1, 2, or 3; b=0, 1, or 2; c=0, 1, or 2; subject to the limitation that a+b+c=3 except when w+x+y+z=0 then a+b+c=4;
M′=(R¹O)_{d}(R²O)ₑR⁴_{f}SiO_{1/2} where the subscripts may have the following values: d=1, 2, or 3; e=0, 1, or 2; f=0, 1, or 2; subject to the limitation that d+e+f=3;
D=R⁵R⁶SiO_{2/2};
T=R⁷SiO_{3/2}; and
Q=SiO_{4/2};
   where R¹ and R² are selected from the group of linear or branched monovalent hydrocarbon radicals having from one to eight carbon atoms and where each R³, R⁴, R⁵, R⁶, and R⁷ is independently selected from the group of monovalent hydrocarbon radicals having from one to sixty carbon atoms; and mixtures thereof.

### FIELD OF THE INVENTION

The present invention concerns novel volatile liquid silanes and siloxanes that function as supports or carriers for cosmetic, cleaning and care products compositions

### BACKGROUND OF THE INVENTION

Volatile siloxanes have been used as evaporable supports in a wide variety of applications ranging from cosmetics to dry cleaning. These siloxanes are excellent solvents for a wide variety of cosmetic ingredients and are mainly used due to the unique feel they provide on skin and hair.

The most common evaporable carriers are cyclic siloxanes such as octamethylcyclotetrasiloxane (D4 or cyclotetrasiloxane), decamethylcyclopentasiloxane (D5 or cyclopentasiloxane) and dodecamethylcyclohexasiloxane (D6 or cyclohexasiloxane). These have 4-6 repeat units and exhibit the volatility profiles that are required for use as evaporable supports/carriers. Although these are the most common supports, there have been recent disclosures on other types of volatile silicon compounds. WO 03/042221 A1, Eversheim, (December 2002) describes a wide variety of substituted short chain siloxanes such as

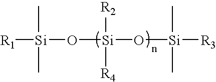

U.S. Pat. No. 4,355,062, Wang et al, issued October 1982 describes additional disclosures of volatile silicone compounds. Other cyclic silicones are described in U.S. Pat. No. 4, 364,837, Pader, issued December 1982. Other volatile linear and cyclic silicon compounds are described in U.S. Pat. No. 5,002,762, Bolich, issued March 1991. A Japanese patent, JP2000063671 describes a linear organosiloxane mixture, production thereof, and cosmetic preparation containing same. Poliniak et al., describe structures such as 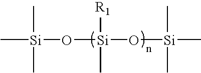
in U.S. Pat. No. 4,376,087 issued in March 1983.

In spite of the fact that a wide variety of silicone compounds are described in the above references, there continues to exist a need for new compostions for cosmetics, cleaning and care products that contain novel evaporable siloxane/silane supports. Therefore, the object of this invention is to describe compositions that exhibit volatility profiles, stability and feel similar to cyclic siloxanes having 4-6 siloxane groups while providing enhanced solvency and compatibility with various cosmetic ingredients.

### SUMMARY OF INVENTION

The present invention relates to compositions made using an evaporable siloxane liquid support or carrier having the molecular formula:
MᵥM′_{w}DₓT_{y}Q_{z}
where the subscripts may have the following values: x=0, 1, 2, 3, or 4; y=0, 1, or 2; z=0, 1, 2, 3, or 4; and v and w may be zero or a positive integer subject to the limitation that v+w≧1 where the components are defined as follows:
M=(R¹O)ₐ(R²O)_{b}R³_{c}SiO_{1/2} where the subscripts may have the following values: a=1, 2, or 3; b=0, 1, or 2; c=0, 1, or 2; subject to the limitation that a+b+c=3 except when w+x+y+z=0 then a+b+c=4;
M′=(R¹O)_{d}(R²O)ₑR⁴_{f}SiO_{1/2} where the subscripts may have the following values: d=1, 2, or 3; e=0, 1, or 2; f=0, 1, or 2; subject to the limitation that d+e+f=3;
D=R⁵R⁶SiO_{2/2};
T=R⁷SiO_{3/2}; and
Q=SiO_{4/2};
   where R¹ and R² are selected from the group of linear or branched monovalent hydrocarbon radicals having from one to eight carbon atoms and where each R³, R⁴, R⁵, R⁶, and R⁷ is independently selected from the group of monovalent hydrocarbon radicals having from one to sixty carbon atoms; and mixtures thereof.

The present invention provides for new formulations for cosmetics, cleaning and care products (collectively retail silicone compositions) prepared by using alkoxy functionalized silanes/siloxanes as the evaporable support/carrier in formulations. These compositions exhibit volatility profiles, stability and feel similar to cyclic siloxanes having 4-6 siloxane groups while providing enhanced solvency and compatibility with various cosmetic ingredients. These alkoxy-functionalized silanes/siloxanes also exhibit very low reactivity.

### DETAILED DESCRIPTION OF THE INVENTION

This present invention provides for formulations for cosmetics, cleaning and care products prepared by using alkoxy functionalized silanes/siloxanes as the evaporable support/carrier in formulations instead of a cyclic siloxane having 4-6 siloxane groups. These compositions exhibit volatility profiles, stability and feel similar to cyclic siloxane having 4-6 siloxane groups while providing low reactivity, enhanced solvency and compatibility with various cosmetic ingredients.

The present invention relates to compositions made using an evaporable or volatile siloxane liquid support or carrier having the molecular formula:
MᵥM′_{w}DₓT_{y}Q_{z}
where the subscripts may have the following values: x=0, 1, 2, 3, or 4; y=0, 1, or 2; z=0, 1, 2, 3, or 4; and v and w may be zero or a positive integer subject to the limitation that v+w≧1 where the components are defined as follows:
M=(R¹O)ₐ(R²O)_{b}R³_{c}SiO_{1/2} where the subscripts may have the following values: a=1, 2, or 3; b=0, 1, or 2; c=0, 1, or 2; subject to the limitation that a+b+c=3 except when w+x+y+z=0 then a+b+c=4;
M′=(R¹O)_{d}(R²O)ₑR⁴_{f}SiO_{1/2} where the subscripts may have the following values: d=1, 2, or 3; e=0, 1, or 2; f=0, 1, or 2; subject to the limitation that d+e+f=3;
D=R⁵R⁶SiO_{2/2};
T=R⁷SiO_{3/2}; and
Q=SiO_{4/2};
   where R¹ and R² are selected from the group of linear or branched monovalent hydrocarbon radicals having from one to eight carbon atoms and where each R³, R⁴, R⁵, R⁶, and R⁷ is independently selected from the group of monovalent hydrocarbon radicals having from one to sixty carbon atoms; and mixtures thereof. A preferred group for R¹ and R² is the group consisting of propyl, i-propyl, butyl, i-butyl and t-butyl.

Examples of alkoxy functionalized silanes/siloxanes include, but are not limited to the following:

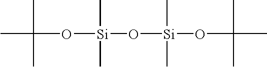

Di-t-butoxytetramethyldisiloxane

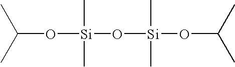

Di-isopropoxy tetramethyldisiloxane

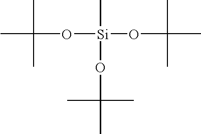

Tri-t-butoxymethylsilane

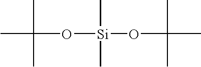

Di-t-butoxydimethylsilane

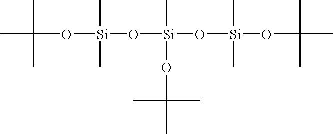

1,3,5-Tri-t-butoxy-pentamethyltrisiloxane

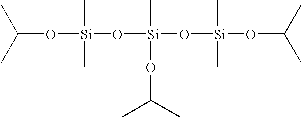

1,3,5-Tri-ti-propoxy-pentamethyltrisiloxane

All of these compounds have a hindered secondary/tertiary alkoxy group that provides for very low reactivity. All these materials have volatility profiles similar or better than cyclic siloxanes having 4-6 siloxane groups. Ones skilled in the art can prepare the compounds described in this invention using well-known procedures.

The personal care applications where the compositions of the present invention may be employed are defined as and include, but are not limited to, deodorants, antiperspirants, antiperspirant/deodorants, shaving products, skin lotions, moisturizers, toners, bath products, cleansing products, hair care products such as shampoos, conditioners, mousses, styling gels, hair sprays, hair dyes, hair color products, hair bleaches, waving products, hair straighteners, manicure products such as nail polish, nail polish remover, nail creams and lotions, cuticle softeners, protective creams such as sunscreen, insect repellent and anti-aging products, color cosmetics such as lipsticks, foundations, face powders, eye liners, eye shadows, blushes, makeup, mascaras, moisturizing preparations; foundations; body and hand preparations; skin care preparations; face and neck preparations; tonics, dressings and other hair grooming aids; hair sprays and aerosol fixatives; fragrance preparations, permanent and non-permanent hair dyes and colors; lipsticks; aftershaves; make-up preparations and soft focus applications; mascaras; night & day skin care preparations; non-coloring hair preparations; deodorants and antiperspirants; eye shadows; tanning preparations; creams and liquids; personal cleansing products; synthetic- and non-synthetic soap bars; hand liquids; nose strips; non-woven applications for personal care; face powders; shampoos, hair conditioners, and shampoos and conditioners; baby lotions; baby baths and shampoos; baby conditioners; shaving preparations; cucumber slices and other skin pads; make-up removers; facial cleansing products; cold creams; sunscreen products; blushes; eyeliners; mousses and spritzes; paste masks and muds; face masks; colognes and toilet waters; hair cuticle coats; nail polishes; deodorant sprays and sticks; roll-on antiperspirant- and deodorant products; shower gels, face- and body washes and other personal care rinse-off products; gels; foam baths; scrubbing cleansers; astringents; nail conditioners; eye shadow sticks; powders for face or eye; lip balms; lip glosses; hair care pump sprays and other non-aerosol sprays; hair-frizz-control gels; hair leave-in conditioners; hair pomades; hair de-tangling products; hair fixatives; hair bleach products; skin lotions; aftershaves; pre-shaves and pre-electric shaves; anhydrous creams and lotions; oil/water,- water/oil,- water/silicone,- oil/silicone, silicone/water,-glycol/silicone,- silicone/glycol,- multiple- and macro- and micro-emulsions; water-resistant creams and lotions; anti-acne preparations; mouth-washes; massage oils; toothpastes; clear gels and sticks; ointment bases; topical wound-healing products; aerosol talcs; barrier sprays; vitamin- and anti-ageing preparations; herbal-extract-preparations; bath salts; bath- and body milks; hair styling aids; hair-, eye-, nail- and skin-soft solid applications; controlled-release formulations; hair conditioning mists; skin care moisturizing mists; skin wipes; pore cleaners; blemish reducers; skin exfoliators; skin desquamation enhancers; skin towelettes & clothes; depilatory preparations; personal care lubricants and nail coloring preparations and other personal care formulations where silicone components have been conventionally added, as well as drug delivery systems for topical application of medicinal compositions that are to be applied to the skin. The uses of the compositions of the present invention are also include cleaning compositions, other products such as waxes, polishes and textiles treatment.

In a preferred embodiment, the personal care composition of the present invention further comprises one or more personal care ingredients. Suitable personal care ingredients include, for example, surfactants and emulsifiers, emollients, moisturizers, humectants, pigments, including pearlescent pigments such as, for example, bismuth oxychloride and titanium dioxide coated mica, colorants, fragrances, biocides, preservatives, antioxidants, anti-microbial agents, anti-fungal agents, antiperspirant agents, exfoliants, hormones, enzymes, medicinal compounds, vitamins, salts, electrolytes, alcohols, polyols, absorbing agents for ultraviolet radiation, botanical extracts, surfactants, silicone oils, organic oils, waxes, film formers, thickening agents such as, for example, gums, acrylic polymers, polyols, fumed silica or hydrated silica, particulate fillers, such as for example, talc, kaolin, starch, modified starch, mica, nylon, polymethylsilsequioxane, clays, such as, for example, bentonite and organo-modified clays.

Suitable personal care compositions are made by combining, in a manner known in the art, such as, for example, by mixing, one or more of the above components with the alkoxy siloxane or silane, preferably in the form of the evaporable silicone compound of the present invention. Suitable personal care compositions may be in the form of a single phase or in the form of an emulsion, including but not limited to oil-in-water, water-in-oil and anhydrous emulsions where the silicone phase may be either the discontinuous phase or the continuous phase, as well as multiple emulsions, such as, for example, oil-in water-in-oil emulsions and water-in-oil-in water-emulsions.

The compositions of the present invention may be utilized as prepared or as one or more components in emulsions. As is generally known, emulsions comprise at least two immiscible phases, one of which is continuous and the other, which is discontinuous. Further, emulsions may be liquids with varying viscosities comprising solids. Additionally, the particle size of the emulsions may render them microemulsions, and when sufficiently small, such microemulsions may be transparent. Further it is also possible to prepare emulsions of emulsions and these are generally known as multiple emulsions.

These primary types of emulsions may be:
1) aqueous emulsions where the discontinuous phase comprises water and the continuous phase comprises the alkoxy siloxane/silanes described in the present invention;
2) aqueous emulsions where the discontinuous phase comprises the alkoxy siloxane/silanes of the present invention and the continuous phase comprises water;
3) non-aqueous emulsions where the discontinuous phase comprises a non-aqueous hydroxylic solvent and the continuous phase comprises the alkoxy siloxane/silanes of the present invention; and
4) non-aqueous emulsions where the continuous phase comprises a non-aqueous hydroxylic organic solvent and the discontinuous phase comprises the alkoxy siloxane/silanes of the present invention.

Non-aqueous emulsions comprising a silicone phase are described in U.S. Pat. Nos. 6,060,546 and 6,271,295 the disclosures of which are herewith and hereby specifically incorporated by reference.

As used herein the term “non-aqueous hydroxylic organic compound” means hydroxyl containing organic compounds as exemplified by but not limited to alcohols, glycols, polyhydric alcohols and polymeric glycols and mixtures thereof that are liquid at room temperature, e.g. about 25° C., and about one atmosphere pressure. The non-aqueous organic hydroxylic solvents are selected from the group consisting of hydroxyl containing organic compounds comprising alcohols, glycols, polyhydric alcohols and polymeric glycols and mixtures thereof that are liquid at room temperature, e.g. about 25° C., and about one atmosphere pressure. Preferably the non-aqueous hydroxylic organic solvent is selected from the group consisting of ethylene glycol, ethanol, propyl alcohol, iso-propyl alcohol, propylene glycol, dipropylene glycol, tripropylene glycol, butylene glycol, iso-butylene glycol, methyl propane diol, glycerin, sorbitol, polyethylene glycol, polypropylene glycol mono alkyl ethers, polyoxyalkylene copolymers and mixtures thereof.

Once the desired form is attained, whether as a silicone only phase, an anhydrous mixture comprising the silicone phase (that may or may not contain so-called non-intended water), a hydrous mixture comprising the silicone phase, a water-in-oil emulsion, an oil-in-water emulsion, or either of the two non-aqueous emulsions or variations thereon, the resulting material is usually a high viscosity cream with good feel characteristics, and high absorbance of volatile solvent. It is capable of being blended into formulations for hair care, skin care, antiperspirants, sunscreens, cosmetics, color cosmetics, insect repellants, vitamin and hormone carriers, fragrance carriers and the like.

Detailed descriptions of suitable product types described before are provided below:

Skin Care Products

In a useful embodiment, a skin care composition comprises the alkoxy siloxane/silane, preferably in the form of the present invention. The skin care composition may, optionally, further include emollients, such as, for example, triglyceride esters, paraffins, wax esters, alkyl or alkenyl esters of fatty acids or polyhydric alcohol esters and one or more of the known components conventionally used in skin care compositions, such as, for example, pigments, herbal- and other extracts, vitamins, such as, for example, Vitamin A, Vitamin C and Vitamin E, sunscreen or sunblock compounds, such as, for example, titanium dioxide, zinc oxide, oxybenzone, octylmethoxy cinnamate, butylmethoxy dibenzoylmethane, p-aminobenzoic acid and octyl dimethyl-p-aminobenzoic acid.

In another useful embodiment, a color cosmetic composition, such as, for example, a lipstick, a makeup or a mascara composition comprises the alkoxy siloxane/silane, preferably in the form of composition of the present invention, and a coloring agent, such as a pigment, a water soluble dye or a liposoluble dye.

In another useful embodiment, the compositions of the present invention are utilized in conjunction with fragrant materials. These fragrant materials may be fragrant compounds, encapsulated fragrant compounds, or fragrance releasing compounds that either the neat compounds or are encapsulated. Particularly compatible with the compositions of the present invention are the fragrance releasing silicon containing compounds as disclosed in U.S. Pat. Nos. 6,046,156; 6,054,547; 6,075,111; 6,077,923; 6,083,901; and 6,153,578; all of which are herein and herewith specifically incorporated by reference.

Deodorants/AP Sticks

In one useful embodiment, an antiperspirant composition comprises the alkoxy siloxane/silane of the present invention and one or more active antiperspirant agents. Suitable antiperspirant agents include, for example, the Category I active antiperspirant ingredients listed in the U.S. Food and Drug Administration's Oct. 10, 1993 Monograph on antiperspirant drug products for over-the-counter human use, such as, for example, aluminum halides, aluminum hydroxyhalides, for example, aluminum chlorohydrate, and complexes or mixtures thereof with zirconyl oxyhalides and zirconyl hydroxyhalides, such as for example, aluminum-zirconium chlorohydrate, aluminum zirconium glycine complexes, such as, for example, aluminum zirconium tetrachlorohydrex gly.

Hair Care Products

The hair care products of the present invention contain the evaporable silicone compounds described above along with a carrier benefiting from the silicon compound. The term “carrier”, as used herein, means one or more compatible compounds suitable for administration to human hair. The term “compatible”, as used herein, means that the evaporable silicone compounds of the present invention are capable of being mixed with the components of the carrier, and with each other, in a manner known to those skilled in the art, such that there is no interaction that would substantially reduce the efficacy of the hair care products under ordinary use conditions.

Carriers suitable for use with the evaporable silicone compounds of the present invention, in applications such as shampoos and cream rinse conditioners to hair, are well known in the art, and their selection can be made by a person skilled in the art. For example, carriers which are suitable are described in more detail in U.S. Pat. No. 4,012,501, Farber, issued Mar. 15, 1977; U.S. Pat. No. 4,223,009, Chakrabarti, issued Sep. 16, 1980; U.S. Pat. No. 4,283,384, Jacquet et al., issued Aug. 11, 1981, U.S. Pat. No. 5,104,646, Bolich Jr., et al., issued Apr. 14, 1992; U.S. Pat. No. 5,436,010, Lau et al., issued Jul. 25, 1995; the disclosures of all these patents being incorporated herein by reference in their entirety.

Shampoo compositions useful with the silicone compounds of this invention utilize conventional components. The shampoos comprise from about 0.1% to about 10% of the volatile silicone compound; from about 5% to about 60% of a synthetic surfactant; and the balance water. Suitable surfactants include sodium lauryl sulfate, sodium laureth sulfate, ammonium lauryl sulfate, ammonium laureth sulfate, potassium lauryl sulfate, potassium laureth sulfate, triethylamine lauryl sulfate, thiethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride lauryl sarcosine, cocoyl sarcosine, ammonium cocoyl sulfate, sodium cocoyl sulfate, potassium cocoyl sulfate, triethanolamine cocoyl sulfate, monoethanolamine cocoyl sulfate, sodium tridecyl benzene sulfonate and sodium dodecyl benzene sulfonate.

Shampoo formulations can also contain various nonessential optional components well known to those skilled in the art, including preservatives, dyes, perfumes, thickeners and viscosity modifiers, pH modifiers, chelating agents, cationic surfactants, and styling or fixative polymers. Such optional ingredients are generally used individually from 0.01% to 10%, preferably from about 0.1% to 5% by weight of the total composition.

Other carriers useful with the present silicon compounds are creme rinses and conditioners. Such carriers are preferably comprised of two essential components, one being a lipid material and the other generally a cationic surfactant material. Such carriers are generally described in the following documents, all incorporated by reference herein: Barry et al., “The Self-Bodying Action of Alkyltrimethylammonium Bromides/Cetostearyl Alcohol Mixed Emulsifiers; Influence of Quaternary Chain Length”, 35, J. of Colloid and Interface Science 689-708 (1971); and Barry et al., “Rheology of Systems Containing Cetomacrogol 1000—Cetostearyl Alcohol, I. Self Bodying Action”, 38, J. of Colloid and Interface Science 616-625 (1972).

Lipid materials suitable for formulation with the compounds of the present invention include acids and acid derivatives, alcohols, esters, ethers, ketones” and amides with carbon chains of from 12 to 22, preferably from 16 to 18, carbon atoms in length. Examples of lipid materials useful herein are disclosed in Bailey's Industrial Oil and Fat Products, (3rd edition, D. Swern, ed. 1979) (incorporated by reference herein).

Creme rinse and conditioner compositions of the present invention generally comprise from about 0.5% to about 12% of the silicon compound, from about 0.5% to about 3% of the lipid vehicle material, and from about 0.2% to about 4% of the cationic surfactant vehicle material.

Cleaning Products

Current dry cleaning technology uses perchloroethylene (“PERC”) or petroleum-based materials as the cleaning solvent. PERC suffers from toxicity and odor issues. The petroleum-based products are not as effective as PERC in cleaning garments.

Cyclic siloxanes have been reported as spot cleaning solutions, see U.S. Pat. No. 4,685,930, and as dry cleaning fluids in dry cleaning machines, see U.S. Pat. No. 5,942,007. Other patents disclose the use of silicone soaps in petroleum solvents, see JP 09299687, and the use of silicone surfactants in super critical carbon dioxide solutions has been reported, see, for example, U.S. Pat. No. 5,676,705 and Chem. Mark. Rep., 15 Dec. 1997, 252(24), p. 15. Non-volatile silicone oils have also been used as the cleaning solvent requiring removal by a second washing with perfluoroalkane to remove the silicone oil, see JP 06327888.

Numerous other patents have issued in which siloxanes or organomodified silicones have been present as addenda in PERC or petroleum based dry cleaning solvents, see, for example, WO 9401510; U.S. Pat. No. 4,911,853.

Metal Ceaning Products

In a preferred embodiment, the composition comprises, based on 100 parts by weight (“pbw”) of the composition, from greater than 90 pbw to 99.99 pbw, more preferably from 92 pbw to 99.9 pbw and even more preferably from 95 pbw to 99.5 pbw of the volatile siloxane and from 0.001 pbw to less than 10 pbw, more preferably from 0.01 pbw to 8 pbw and even more preferably from 0.1 pbw to 5 pbw of the surfactants. The surfactants are selected from the classes of nonionic, cationic, anionic and amphoteric surfactants.

Compounds suitable for use as the nonionic surfactant of the present invention are those that carry no discrete charge when dissolved in aqueous media. Nonionic surfactants are generally known in the art and include, for example, alkanol amides (such as, for example, coco, lauric, oleic and stearic monoethanolamides, diethanolamides and monoisopropanolamides), amine oxides (such as, for example, polyoxyethylene ethanolamides and polyoxyethylene propanolamides), polyalkylene oxide block copolymers (such as, for example, poly(oxyethylene-co-oxypropylene)), ethoxylated alcohols, (such as, for example, isostearyl polyoxyethylene alcohol, lauryl, cetyl, stearyl, oleyl, tridecyl, trimethylnonyl, isodecyl, tridecyl), ethoxylated alkylphenols (such as, for example, nonylphenol), ethoxylated amines and ethoxylated amides, ethoxylated fatty acids, ethoxylated fatty esters and ethoxylated fatty oils (such as, for example, mono- and diesters of acids such as lauric, isostearic, pelargonic, oleic, coco, stearic, and ricinoleic, and oils such as castor oil and tall oil), fatty esters, fluorocarbon containing materials, glycerol esters (such as, for example, glycerol monostearate, glycerol monolaurate, glycerol dilaurate, glycerol monoricinoleate, and glycerol oleate), glycol esters (such as, for example, propylene glycol monostearate, ethylene glycol monostearate, ethylene glycol distearate, diethylene glycol monolaurate, diethylene glycol monolaurate, diethylene glycol monooleate, and diethylene glycol stearate), lanolin-based surfactants, monoglycerides, phosphate esters, polysaccharide ethers, propoxylated fatty acids, propoxylated alcohols, and propoxylated alkylphenols, protein-based organic surfactants, sorbitan-based surfactants (such as, for example, sorbitan oleate, sorbitan monolaurate, and sorbitan palmitate), sucrose esters and glucose esters, and thio- and mercapto-based surfactants.

The uses of the compositions of the present invention are not restricted to personal care and cleaning compositions, other products such as waxes, polishes and textiles treated with the compositions of the present invention are also contemplated; thus the phrase retail silicone composition is defined to include all the applications disclosed herein. One of the advantages of using the compositions of the present invention are that the evaporable siloxanes of the present invention provide an improved compatibility for the other lipophilic or organic components or compounds utilized in the compositions of the consumer products described herein. Compatibility is defined for a formulation either as no observable phase separation or syneresis of formulated components within the first 300 hours at 50° C. or the formulation remains homogenous during the application of the formulation (or product) with no perceivable separation of the organic compound or compounds dispersed or dissolved in the formulation. The organic compounds utilized in the formulations or consumer products described herein have an equal or greater solubility in the evaporable siloxanes (or silicone compositoins) of the present invention than such compounds possess relative to a cyclic siloxane of equal or greater molecular weight relative to the molecular weight of the siloxane or silicone composition. Organic compounds satisfying this criterion may be selected from the group consisting of butylmethoxydibenzoylmethane, 4-methyl benzylidene camphor, 4-methyl benzylidene campho, benzophenone-4, benzophenone-3, petrolatum, propylene glycol, and cetyl alcohol

### EXAMPLES

The following examples are to illustrate the invention and are not to be construed as limiting the claims.

### Synthesis Examples

A. Di-t-butoxytetramethyldisiloxane: A 1 L round-bottomed flask equipped with overhead stirrer, thermometer, reflux condenser with nitrogen inlet, and addition funnel was charged with tetramethyldisiloxane (241.1 g, 1.795 mol). The siloxane was stirred and brought to 60° C. under N₂. A solution of tris(pentafluorophenyl)borane (0.092 g, 0.180 mmol) in tert-butanol (266.1 g, 3.591 mol) was charged to the addition funnel and added dropwise to the siloxane. Vigorous evolution of H₂ gas was observed, and the reaction temperature increased to 65-70° C. After addition of half of the tert-butanol solution (˜3 h), gas evolution had slowed, and the reaction temperature was brought to 85° C. The addition was continued at this temperature, and was complete within another 2.5 h. The reaction was brought to reflux at 95° C. for 2.5 h, and then allowed to cool to RT. FT-IR analysis of the reaction mixture indicated no hydride present, some excess alcohol. The reaction mixture was distilled under vacuum to yield a clear fluid, that was found to be >96% purity by GC.

B. Di-isopropoxytetramethyldisiloxane: A 500 mL round-bottomed flask equipped with a thermometer, addition funnel and reflux condenser with nitrogen inlet was charged with isopropanol (95.2 g, 1.584 mol) and Karstedt's catalyst (10.9 wt. % solution of Pt(0) in divinyltetramethyldisiloxaneⁱ; 71 mg). The solution was stirred and brought to 60° C. under N₂. Tetramethyldisiloxane (106.4 g, 0.792 mol) was charged to the addition funnel and added dropwise to the isopropanol. During addition, gas evolution and an exotherm were noted, and the addition rate was slowed to maintain a reaction temperature of 70-75° C. After complete addition (2.5 h), the reaction temperature was brought to 85° C. and maintained for 12 h. GC analysis of the reaction mixture indicated both the single addition product (M′M^{H}) and isopropanol remaining, so additional catalyst (36 mg) was added, and the reaction was maintained at reflux (85° C.) for 2 h. FT-IR analysis of the reaction mixture indicated no hydride or alcohol present. The reaction mixture was distilled under vacuum, and the product was isolated to yield a clear fluid, that was found to be >75% purity by GC.

C. Tri-t-butoxymethylsilane: A 500 mL round-bottomed flask equipped with overhead stirrer, thermometer, reflux condenser with nitrogen inlet, and addition funnel was charged with 100 g of toluene, 63 g of tert-butanol, 31.1 g of trichlorosilane and 53 g of pyridene. The siloxane was stirred and brought to reflux under N₂ for 36 h. At this point, another 30 g pyridene was added to the reaction and held to reflux for 12 h. GC analysis showed that the conversion to tri-t-butoxymethylsilane was 90%. The salts precipitated were then filtered and 100 g of toluene was added to dilute the solution. This diluted was then washed with 300 mL of water, followed by 300 mL of 2% HCl, and finally with 300 mL of water in a separatory funnel. The organic layer was then dried over sodium sulfate. The toluene was removed via rotary evaporation. The reaction mixture was distilled under vacuum to yield a clear fluid, that was found to be >95% purity by Gas Chromatography.

D. Di-t-butoxydimethylsilane: A 1 L round-bottomed flask equipped with overhead stirrer, thermometer, reflux condenser with nitrogen inlet, and addition funnel was charged with 200 mL of hexane, 82 g of tert-butanol (1.1 mol) and 110 g of triethylamine (1.1 mol). The siloxane was stirred and brought to 50° C. under N₂. To this solution was added 64.5 g of dichloro dimethylsilane using an addition funnel and added dropwise. After complete addition, the reaction was held at reflux temperature overnight. The salts precipitated were filtered and the hexane solution was washed with 200 mL of 0.1 N HCl followed by 200 mL water in a separatory funnel. The organic layer was then dried over sodium sulfate. The hexane was then separated via rotary evaporation. The reaction mixture was distilled under vacuum to yield a clear fluid, that was found to have >98% purity by Gas Chromatography.

### Formulation Examples

### Example 1

### Antiperspirant Solid

TABLE 1
Formulation E1a&b:

| | Part/Wt | |
|---|---|---|
| Ingredient | (%) | Function |
| Di-t-butoxytetramethyldisiloxane | X | Actives carrier |
| | | Dry/Non-greasy |
| | | feel |
| Dimethicone (SF96-100) (1) | 5.0 | Emollient/Anti- |
| | | whitening |
| Stearyl Alcohol | 19.0 | Structuring agent |
| Hydrogenated Castor Oil (mp 70° C.) | 3.0 | Structuring agent |
| Talc | 4.0 | Smooth feel |
| Glyceryl Stearate (and) PEG-100 Stearate (2) | 2.0 | Suspension |
| Aluminum Zirconium Tetrachlorohydrex Gly | Y | Antiperspirant |
| | | active |

With X = 49.00% (a) and 51.00% (b), and with Y = 18% (a) and 16% (b), respectively.

Procedure:

1. Di-t-butoxytetramethyldisiloxane, dimethicone and stearyl alcohol were mixed together.

2. To the above mixture was added antiperspirant active, talc and glyceryl stearate (and) PEG-100 stearate.

3. This was then heated to 75° C. and stirred with moderate agitation until all wax had melted.

4. The hydrogenated castor oil was pre-melted and added to mixture as a liquid and stirred for 15 minutes.

5. This mixture was then cooled to 55° C. with continued mixing and poured into a container.

Trade Names/Suppliers: (1) GE Silicones(2) Uniqema, Inc.

### Comparative Example

The experiment in example 1 was repeated wherein di-t-butoxytetramethyldisiloxane was replaced with cyclopentasiloxane (Formulation CE1), using the same process conditions as outlined above:

TABLE 2

| | Part/Wt | |
|---|---|---|
| Ingredient | (%) | Function |
| Cyclopentasiloxane | 45.0 | Actives carrier |
| | | Dry/Non-greasy |
| | | feel |
| Dimethicone (SF96-100) (1) | 5.0 | Emollient/Anti- |
| | | whitening |
| Stearyl Alcohol | 19.0 | Structuring agent |
| Hydrogenated Castor Oil (mp 70° C.) | 3.0 | Structuring agent |
| Talc | 4.0 | Smooth feel |
| Glyceryl Stearate (and) PEG-100 Stearate (2) | 2.0 | Suspension |
| Aluminum Zirconium Tetrachlorohydrex Gly | 22.0 | Antiperspirant |
| | | active |

Results

Example 1 provides an antiperspirant stick formulation wherein the di-t-butoxytetramethyldisiloxane acts as a fugitive carrier for the antiperspirant active, thus providing a smooth, dry, non-greasy feel providing a stable matrix for actives integration, stick integrity and strength. The di-t-butoxytetramethyldisiloxane- and cyclopentasiloxane-containing formulations exhibit no difference in the visual stability test performance:

TABLE 3

| | 1 Week | 1 Week | 2 weeks | 2 Weeks | 4 Weeks | 4 Weeks |
|---|---|---|---|---|---|---|
| | RT | 50 C. | RT | 50 C. | RT | 50 C. |
| E1a&b | Ok | ok | ok | ok | ok | ok |
| CE1 | Ok | ok | ok | ok | ok | ok |

### Example 2

### Skin Lotion

TABLE 4
Formulation E2

| | Part/Wt | |
|---|---|---|
| Ingredient | (%) | Function |
| PART A | | |
| Cyclopentasiloxane (and) PEG/PPG- | 2.5 | Emulsifier |
| 20/15Dimethicone (SF1540) (1) | | |
| Di-t-butoxytetramethyldisiloxane | 16.0 | Emollient |
| Cyclopentasiloxane (and) Dimethicone | 7.5 | Emollient/Film former |
| (SF1214) (1) | | |
| PART B | | |
| Glycerin | 3.0 | Humectant |
| Sodium Chloride | 1.0 | Stabilizer |
| Polysorbate-80 | 0.2 | Emulsifier |
| Quaternium-15 | 0.1 | Preservative |
| Deionized Water | 69.7 | Diluent |

Procedure:

1. The Part A ingredients were combined together in order shown, thoroughly mixing each component until homogeneous before adding the next ingredient.

2. All Part B ingredients were mixed together.

3. Slowly, the Part B mixture was added to Part A with good mixing. The agitation was gradually increased high shear as mixture thickened. The agitation was continues for a further 10 minutes, when the mixture became very thick.

4. This was then submitted to a blender for 2 minutes.

Trade Names/Suppliers: (1) GE Silicones.

### Comparative Example 2

Here, cyclopentasiloxane replaces di-t-butoxytetramethyldisiloxane (Formulation CE2) using the same process conditions as outlined above:

TABLE 5

| | Part/Wt | |
|---|---|---|
| Ingredient | (%) | Function |
| PART A | | |
| Cyclopentasiloxane (and) PEG/PPG- | 2.5 | Emulsifier |
| 20/15Dimethicone (SF1540) (1) | | |
| Cyclopentasiloxane | 16.0 | Emollient |
| Cyclopentasiloxane (and) Dimethicone | 7.5 | Emollient/Film former |
| (SF1214) (1) | | |
| PART B | | |
| Glycerin | 3.0 | Humectant |
| Sodium Chloride | 1.0 | Stabilizer |
| Polysorbate-80 | 0.2 | Emulsifier |
| Quaternium-15 | 0.1 | Preservative |
| Deionized Water | 69.7 | Diluent |

Results:

Example 2 provides a skin lotion example formulation wherein the di-t-butoxytetramethyldisiloxane acts as skin sensory enhancer and emollient, providing a silky-light, yet substantive non-tacky skin feel. The di-t-butoxytetramethyldisiloxane- and cyclopentasiloxane-containing formulations exhibit no difference in the visual stability test performance:

TABLE 6

| | | | | | 4 | |
|---|---|---|---|---|---|---|
| | 1 Week | 1 Week | 2 weeks | | Weeks | 4 Weeks |
| | RT | 50C | RT | 2 Weeks 50C | RT | 50C |
| E2 | Ok | ok | ok | some syneresis | ok | some |
| | | | | | | syneresis |
| CE2 | Ok | ok | ok | some syneresis | ok | some |
| | | | | | | syneresis |

### Example 3

### Light Satin Lotion

TABLE 7
Formulation E3:

| | Part/Wt | |
|---|---|---|
| Ingredient | (%) | Function |
| PART A | | |
| Sorbitan Oleate | 0.60 | Co-emulsifier |
| Cyclopentasiloxane (and) PEG/PPG-20/15 | 2.50 | Emulsifier |
| Dimethicone (SF1540) (1) | | |
| Cyclopentasiloxane (and) C30-45 Alkyl | 7.50 | Substantive silky |
| Cetearyl Dimethicone Crosspolymer | | feel |
| (Velvesil ® 125) (1) | | |
| Di-t-butoxytetramethyldisiloxane | 16.50 | Emollient |
| PART B | | |
| Butylene Glycol | 1.00 | Humectant |
| Sodium Chloride | 1.00 | Stabilizer |
| Quaternium-15 | 0.10 | Preservative |
| Water | 70.8 | Diluent |

Velvesil is a registered trademark of General Electric Company
Procedure:

1. Part A ingredients were combined in the order shown, thoroughly mixing each component until homogeneous before adding next ingredient.

2. All ingredients of Part B were mixed together and stirred well until homogeneous.

3. Slowly, the Part B mixture was added to Part A with good mixing. Gradually, the agitation was increased to high shear as the mixture thickened. The agitation was continued for 20 minutes.

4. This was then submitted to a blender for 2 minutes.

Trade Names/Suppliers: (1) GE Silicones

### Comparative Example 3

Here, cyclopentasiloxane replaces di-t-butoxytetramethyldisiloxane (Formulation CE3) using the same process conditions as outlined above:

TABLE 8

| | Part/Wt | |
|---|---|---|
| Ingredient | (%) | Function |
| PART A | | |
| Sorbitan Oleate | 0.60 | Co-emulsifier |
| Cyclopentasiloxane (and) PEG/PPG-20/15 | 2.50 | Emulsifier |
| Dimethicone (SF1540) (1) | | |
| Cyclopentasiloxane (and) C30-45 Alkyl | 7.50 | Substantive silky |
| Cetearyl Dimethicone Crosspolymer | | feel |
| (Velvesil ® 125) (1) | | |
| Cyclopentasiloxane | 16.50 | Emollient |
| PART B | | |
| Butylene Glycol | 1.00 | Humectant |
| Sodium Chloride | 1.00 | Stabilizer |
| Quaternium-15 | 0.10 | Preservative |
| Water | 70.8 | Diluent |

Results:

Example 3 provides a water-in-oil skin lotion example formulation wherein the di-t-butoxytetramethyldisiloxane acts as skin sensory enhancer and emollient, providing a silky-light, yet substantive non-tacky skin feel. The di-t-butoxytetramethyldisiloxane- and cyclopentasiloxane-containing formulations exhibit no difference in the visual stability test performance:

TABLE 9

| | 1 Week | 1 Week | 2 weeks | 2 Weeks | 4 Weeks | 4 Weeks |
|---|---|---|---|---|---|---|
| | RT | 50 C. | RT | 50 C. | RT | 50 C. |
| E3 | ok | ok | ok | ok | ok | ok |
| CE3 | ok | ok | ok | ok | ok | ok |

### Example 4

### Sheer Silky Make-Up Foundation

TABLE 10
Formulation E4:

| | Part/Wt | |
|---|---|---|
| Ingredient | (%) | Function |
| PART A | | |
| Cyclopentasiloxane (and) PEG/PPG-20-15 | 5.12 | Emulsifier |
| Dimethicone (SF1540) (1) | | |
| Cyclopentasiloxane (and) Dimethicone/ | 3.0 | Smooth, silky feel |
| Vinyl Dimethicone Crosspolymer | | |
| (SFE839) (1) | | |
| C30-45 Alkyl Dimethicone (SF1642) (1) | 2.0 | Thickener/Emollient |
| Di-t-butoxytetramethyldisiloxane | 24.0 | Emollient |
| Phenyl Trimethicone (SF1550) (1) | 3.0 | Emollient |
| Titanium Dioxide (2) | 7.6 | Pigment |
| Yellow Iron Oxides (3) | 2.8 | Pigment |
| Red Iron Oxides (3) | 1.3 | Pigment |
| Black Iron Oxides (3) | 0.18 | Pigment |
| Sorbitan Oleate | 0.5 | Emulsifier |
| PART B | | |
| Deionized Water | 49.3 | Diluent |
| Polysorbate-20 | 0.2 | Emulsifier |
| Sodium Chloride | 1.0 | Stabilizer |

Procedure:

1. The ingredients of Part A were combined, in order shown, thoroughly mixing each component until homogenous before adding the next ingredient. This was then heated to 60° C. and mixed until SF1642 is dissolved.

2. In a separate vessel, the ingredients of Part B were combined in the order shown.

3. Slowly Part B was added to Part A with good mixing.

4. The mixture was poured into suitable containers. Trade Names/Suppliers: (1) GE Silicones(2) IN80C®, Kobo Products(3) Kobo Products

### Comparative Example 4

Here, cyclopentasiloxane replaces di-t-butoxytetramethyldisiloxane (Formulation CE4) using the same process conditions as outlined above:

TABLE 11

| | Part/Wt | |
|---|---|---|
| Ingredient | (%) | Function |
| PART A | | |
| Cyclopentasiloxane (and) PEG/PPG-20-15 | 5.12 | Emulsifier |
| Dimethicone (SF1540) (1) | | |
| Cyclopentasiloxane (and) Dimethicone/Vinyl | 3.0 | Smooth, silky feel |
| Dimethicone Crosspolymer (SFE839) (1) | | |
| C30-45 Alkyl Dimethicone (SF1642) (1) | 2.5 | Thickener/ |
| | | Emollient |
| Cyclopentasiloxane | 24.0 | Emollient |
| Phenyl Trimethicone (SF1550) (1) | 3.0 | Emollient |
| Titanium Dioxide (2) | 7.6 | Pigment |
| Yellow Iron Oxides (3) | 2.8 | Pigment |
| Red Iron Oxides (3) | 1.3 | Pigment |
| Black Iron Oxides (3) | 0.18 | Pigment |
| Sorbitan Oleate | 0.5 | Emulsifier |
| PART B | | |
| Deionized Water | 48.8 | Diluent |
| Polysorbate-20 | 0.2 | Emulsifier |
| Sodium Chloride | 1.0 | Stabilizer |

Results:

Example 4 provides a foundation example formulation wherein the di-t-butoxytetramethyldisiloxane acts as an excellent emollient, providing smooth, luxurious silky feel and spreadibility of the dyes. The di-t-butoxytetramethyldisiloxane- and cyclopentasiloxane containing formulations exhibit no difference in the visual stability test performance.

TABLE 12

| | 1 Week | 1 Week | 2 weeks | 2 Weeks | 4 Weeks | 4 Weeks |
|---|---|---|---|---|---|---|
| | RT | 50 C. | RT | 50 C. | RT | 50 C. |
| E4 | Ok | syneresis | ok | syneresis | ok | syneresis |
| CE4 | Ok | syneresis | ok | syneresis | ok | syneresis |

It is notable that syneresis in this product form is accepted by the consumer (“liquid foundations”), hence the phenomena observed above are not unusual. In both cases, the emulsions are readily re-mixable through simple shaking, and hence reusable and commercially acceptable.

### Example 5

### Protective Facial Sunscreen with Superior Substantivity

Formulation E5:

| | Part/Wt | |
|---|---|---|
| Ingredient | (%) | Function |
| PART A | | |
| Stearic Acid | 2.50 | Emulsifier |
| Cetyl Alcohol | 1.80 | Thickener/Emulsifier |
| DEA Cetyl Phosphate (1) | 2.50 | Emulsifier |
| Diisostearoyl Trimethylolpropane | 5.00 | Emollient/Film-former |
| Siloxy Silicate (SF1318) (2) | | UV absorber |
| Octyl Methoxycinnamate | 7.00 | Emollient |
| Di-t-butoxytetramethyldisiloxane | 5.00 | Humectant |
| PART B | | |
| Glycerin | 4.00 | Preservative |
| Quaternium-15 | 0.10 | Thickener/Stabilizer |
| Xanthan Gum | 0.25 | Diluent |
| Water | 71.85 | |

Procedure:

1. Part A and B were mixed in separate containers to 85-90° C. with agitation.

2. Part A contents were added to Part B with high shear agitation.

3. Cool to room temperature with continued mixing.

Trade Names/Suppliers: (1) Amphisol™, Givaudan(2) GE Silicones

### Comparative Example 5

Here, cyclopentasiloxane replaces di-t-butoxytetramethyldisiloxane (Formulation CE5) using the same process conditions as outlined above:

| | Part/Wt | |
|---|---|---|
| Ingredient | (%) | Function |
| PART A | | |
| Stearic Acid | 2.50 | Emulsifier |
| Cetyl Alcohol | 1.80 | Thickener/Emulsifier |
| DEA Cetyl Phosphate (1) | 2.50 | Emulsifier |
| Diisostearoyl Trimethylolpropane | 5.00 | Emollient/Film-former |
| Siloxy Silicate (SF1318) (2) | | |
| Octyl Methoxycinnamate | 7.00 | UV absorber |
| Cyclopentasiloxane | 5.00 | Emollient |
| PART B | | |
| Glycerin | 4.00 | Humectant |
| Quaternium-15 | 0.10 | Preservative |
| Xanthan Gum | 0.25 | Thickener/Stabilizer |
| Water | 71.85 | Diluent |

Results:

Example 5 provides an oil-in-water skin cream example formulation wherein the di-t-butoxytetramethyldisiloxane acts as skin sensory enhancer and emollient, providing a silky-light, yet substantive non-tacky skin feel. The di-t-butoxytetramethyldisiloxane- and cyclopentasiloxane-containing formulations exhibit no difference in the visual stability test performance.

| | 1 Week | 1 Week | 2 weeks | 2 Weeks | 4 Weeks | 4 Weeks |
|---|---|---|---|---|---|---|
| | RT | 50 C. | RT | 50 C. | RT | 50 C. |
| E5 | ok | ok | ok | ok | ok | ok |
| CE5 | ok | ok | ok | ok | ok | ok |

### Example 6

### Light Satin Lotion

Formulation E6:

| | Part/Wt | |
|---|---|---|
| Ingredient | (%) | Function |
| PART A | | |
| Sorbitan Oleate | 0.60 | Co- |
| | | emulsifier |
| Cyclopentasiloxane (and) PEG/PPG-20/15 | 2.50 | Emulsifier |
| Dimethicone (SF1540) (1) | | |
| Cyclopentasiloxane (and) C30-45 Alkyl Cetearyl | 7.50 | Substantive |
| Dimethicone Crosspolymer (Velvesil ® 125) (1) | | silky feel |
| Tri-t-butoxymethylsilane | 16.50 | Emollient |
| PART B | | |
| Butylene Glycol | 1.00 | Humectant |
| Sodium Chloride | 1.00 | Stabilizer |
| Quaternium-15 | 0.10 | Preservative |
| Water | 70.8 | Diluent |

Procedure:

1. Part A ingredients were combined in the order shown, thoroughly mixing each component until homogeneous before adding next ingredient.

2. All ingredients of Part B were mixed together and stirred well until homogeneous.

3. Slowly, the Part B mixture was added to Part A with good mixing. Gradually, the agitation was increased to high shear as the mixture thickened. The agitation was contined for 20 minutes.

4. This was then milled on the homogenizer for 2 minutes.

Trade Names/Suppliers: (1) GE Silicones

### Comparative Example 6

Here, cyclopentasiloxane replaces tri-t-butoxymethylsilane (Formulation CE6) using the same process conditions as outlined above:

| | Part/Wt | |
|---|---|---|
| Ingredient | (%) | Function |
| PART A | | |
| Sorbitan Oleate | 0.60 | Co- |
| | | emulsifier |
| Cyclopentasiloxane (and) PEG/PPG-20/15 | 2.50 | Emulsifier |
| Dimethicone (SF1540) (1) | | |
| Cyclopentasiloxane (and) C30-45 Alkyl Cetearyl | 7.50 | Substantive |
| Dimethicone Crosspolymer (Velvesil ® 125) (1) | | silky feel |
| Cyclopentasiloxane | 16.50 | Emollient |
| PART B | | |
| Butylene Glycol | 1.00 | Humectant |
| Sodium Chloride | 1.00 | Stabilizer |
| Quaternium-15 | 0.10 | Preservative |
| Water | 70.8 | Diluent |

Results:

Example 6 provides another skin lotion example formulation wherein the tri-t-butoxymethylsilane acts as skin sensory enhancer and emollient, providing a silky-light, yet substantive non-tacky skin feel. The tri-t-butoxymethylsilane- and cyclopentasiloxane-containing formulations exhibit no difference in the visual stability test performance.

| | 1 Week | 1 Week | 2 Weeks | 2 weeks | 4 Weeks | 4 Weeks |
|---|---|---|---|---|---|---|
| | RT | 50 C. | RT | 50 C. | RT | 50 C. |
| E6 | ok | ok | ok | Ok | ok | ok |
| CE6 | ok | ok | ok | Ok | ok | ok |

### Example 7

### Light Satin Lotion

Formulation E7:

| | Part/Wt | |
|---|---|---|
| Ingredient | (%) | Function |
| PART A | | |
| Sorbitan Oleate | 0.60 | Co- |
| | | emulsifier |
| Cyclopentasiloxane (and) PEG/PPG-20/15 | 2.50 | Emulsifier |
| Dimethicone (SF1540) (1) | | |
| Cyclopentasiloxane (and) C30-45 Alkyl Cetearyl | 7.50 | Substantive |
| Dimethicone Crosspolymer (Velvesil ® 125) (1) | | silky feel |
| Di-t-butoxydimethylsilane | 16.50 | Emollient |
| PART B | | |
| Butylene Glycol | 1.00 | Humectant |
| Sodium Chloride | 1.00 | Stabilizer |
| Quaternium-15 | 0.10 | Preservative |
| Water | 70.8 | Diluent |

Procedure:

1. Part A ingredients were combined in the order shown, thoroughly mixing each component until homogeneous before adding next ingredient.

2. All ingredients of Part B were mixed together and stirred well until homogeneous.

3. Slowly, the Part B mixture was added to Part A with good mixing. Gradually, the agitation was increased to high shear as the mixture thickened. The agitation was continued for 20 minutes.

4. This was then submitted to a blender for 2 minutes.

Trade Names/Suppliers: (1) GE Silicones

### Comparative Example 7

Here, cyclopentasiloxane replaces di-t-butoxydimethylsilane (Formulation CE7) using the same process conditions as outlined above:

| | Part/Wt | |
|---|---|---|
| Ingredient | (%) | Function |
| PART A | | |
| Sorbitan Oleate | 0.60 | Co- |
| | | emulsifier |
| Cyclopentasiloxane (and) PEG/PPG-20/15 | 2.50 | Emulsifier |
| Dimethicone (SF1540) (1) | | |
| Cyclopentasiloxane (and) C30-45 Alkyl Cetearyl | 7.50 | Substantive |
| Dimethicone Crosspolymer (Velvesil ® 125) (1) | | silky feel |
| Cyclopentasiloxane | 16.50 | Emollient |
| PART B | | |
| Butylene Glycol | 1.00 | Humectant |
| Sodium Chloride | 1.00 | Stabilizer |
| Quaternium-15 | 0.10 | Preservative |
| Water | 70.8 | Diluent |

Results:

Example 7 provides another skin lotion example formulation wherein the di-t-butoxydimethylsilane acts as skin sensory enhancer and emollient, providing a silky-light, yet substantive non-tacky skin feel. The di-t-butoxydimethylsilane- and cyclopentasiloxane-containing formulations exhibit no difference in the visual stability test performance.

| | 1 Week RT | 1 Week 50C | 2 weeks RT | 2 Weeks 50C |
|---|---|---|---|---|
| E7 | ok | ok | ok | ok |
| CE7 | ok | ok | ok | ok |

### Example 8

### Light Satin Lotion

Formulation E8:

| | Part/Wt | |
|---|---|---|
| Ingredient | (%) | Function |
| PART A | | |
| Sorbitan Oleate | 0.60 | Co- |
| | | emulsifier |
| Cyclopentasiloxane (and) PEG/PPG-20/15 | 2.50 | Emulsifier |
| Dimethicone (SF1540) (1) | | |
| Cyclopentasiloxane (and) C30-45 Alkyl Cetearyl | 7.50 | Substantive |
| Dimethicone Crosspolymer (Velvesil ® 125) (1) | | silky feel |
| Di-isopropoxy tetramethyldisiloxane | 16.50 | Emollient |
| PART B | | |
| Butylene Glycol | 1.00 | Humectant |
| Sodium Chloride | 1.00 | Stabilizer |
| Quaternium-15 | 0.10 | Preservative |
| Water | 70.8 | Diluent |

Procedure:

1. Part A ingredients were combined in the order shown, thoroughly mixing each component until homogeneous before adding next ingredient.

2. All ingredients of Part B were mixed together and stirred well until homogeneous.

3. Slowly, the Part B mixture was added to Part A with good mixing. Gradually, the agitation was increased to high shear as the mixture thickened. The agitation was continued for 20 minutes.

4. This was then submitted to a blender for 2 minutes.

Trade Names/Suppliers: (1) GE Silicones

### Comparative Example 8

Here, cyclopentasiloxane replaces di-isopropoxy tetramethyldisiloxane (Formulation CE8) using the same process conditions as outlined above:

| | Part/Wt | |
|---|---|---|
| Ingredient | (%) | Function |
| PART A | | |
| Sorbitan Oleate | 0.60 | Co- |
| | | emulsifier |
| Cyclopentasiloxane (and) PEG/PPG-20/15 | 2.50 | Emulsifier |
| Dimethicone (SF1540) (1) | | |
| Cyclopentasiloxane (and) C30-45 Alkyl Cetearyl | 7.50 | Substantive |
| Dimethicone Crosspolymer (Velvesil ® 125) (1) | | silky feel |
| Cyclopentasiloxane | 16.50 | Emollient |
| PART B | | |
| Butylene Glycol | 1.00 | Humectant |
| Sodium Chloride | 1.00 | Stabilizer |
| Quaternium-15 | 0.10 | Preservative |
| Water | 70.8 | Diluent |

Results:

Example 8 provides another skin lotion example formulation wherein the di-isopropoxy tetramethyldisiloxane acts as skin sensory enhancer and emollient, providing a silky-light, yet substantive non-tacky skin feel. Both the di-isopropoxytetramethyldisiloxane- and cyclopentasiloxane- containing formulations exhibit no difference in the visual stability test performance.

| | 1 Week RT | 1 Week 50C | 2 weeks RT | 2 Weeks 50C |
|---|---|---|---|---|
| E8 | Ok | ok | ok | ok |
| CE8 | ok | ok | ok | Ok |

### Example 9

### Hair Cuticle Coat

Formulation E9:

| | Part/Wt | |
|---|---|---|
| Ingredient | (%) | Function |
| PART A | | |
| Di-t-butoxytetramethyldisiloxane | 55.03 | Carrier |
| Dimethicone (1) | 9.97 | Conditioning/Shine |
| PART B | | |
| Isohexadecane | 33.00 | Carrier/Dry time |
| Octyl Methoxycinnamate | 2.00 | UV absorber |

Procedure:

1. Dimethicone was dissolved in di-t-butoxytetramethyldisiloxane with stirring at 75° C. for 6 hours.

2. All ingredients of Part B were mixed together and stirred well until homogeneous.

3. Slowly, the Part B mixture was added to Part A with good mixing. The agitation was continued for 30 minutes.

Trade Names/Suppliers: (1) GE Silicones

### Comparative Example 9

Here, cyclopentasiloxane replaces di-t-butoxytetramethyldisiloxane (Formulation CE9) using the same process conditions as outlined above:

| | Part/Wt | |
|---|---|---|
| Ingredient | (%) | Function |
| PART A | | |
| Cyclopentasiloxane | 55.03 | Carrier |
| Dimethicone (1) | 9.97 | Conditioning/Shine |
| PART B | | |
| Isohexadecane | 33.00 | Carrier/Dry time |
| Octyl Methoxycinnamate | 2.00 | UV absorber |

Results:

Example 9 provides a hair cuticle coat example formulation wherein the di-t-butoxytetramethyldisiloxane acts as a sensory enhancer and emollient, providing a silky-light, yet substantive non-tacky skin feel. The di-t-butoxytetramethyldisiloxane- and cyclopentasiloxane- containing formulations exhibit no difference in the visual stability test performance.

### Example 10

### Hair Conditioner for Damaged Hair

Formulation E10:

| | Part/Wt | |
|---|---|---|
| Ingredient | (%) | Function |
| PART A | | |
| Ceteareth-20 | 1.00 | Emulsifier |
| Stearyl Alcohol | 2.00 | Emulsifier |
| Quaternium-15 | 1.4 | Condioner |
| Di-t-butoxytetramethyldisiloxane | 3.8 | Wet-combing/Quick |
| | | dry |
| PART B | | |
| Water | 89.7 | Diluent |
| PART C | | |
| Amodimethicone (and) Isolaureth-6 (and) | 2 | Conditioner/Shine |
| Glycerin (and) Octoxynol-40 (1) | | |
| Methylchloroisothiazolinone (and) | 0.1 | Preservative |
| Methylisothiazolinone | | |

Procedure:

1. Preheat phases A and B in separate vessels to 75 C.

2. Add B to A under moderate agitation.

3. Cool resulting mixture to 40-50 C

4. Add carefully Amodimethicone (and) Isolaureth-6 (and) Glycerin (and) Octoxynol-40, and then Methylchloroisothiazolinone (and) Methylisothiazolinone.

5. Cool to room temperature.

Trade Names/Suppliers: (1) GE Silicones

### Comparative Example 10

Here, cyclopentasiloxane replaces di-t-butoxytetramethyldisiloxane (Formulation CE10) using the same process conditions as outlined above:

| | Part/Wt | |
|---|---|---|
| Ingredient | (%) | Function |
| PART A | | |
| Ceteareth-20 | 1.00 | Emulsifier |
| Stearyl Alcohol | 2.00 | Emulsifier |
| Quaternium-15 | 1.4 | Conditioner/Preservative |
| Cyclopentasiloxane (1) | 3.8 | Wet-combing/Quick dry |
| PART B | | |
| Water | 89.7 | Diluent |
| PART C | | |
| Amodimethicone | 2 | Conditioner/Shine |
| (and) Isolaureth-6 (and) | | |
| Glycerin (and) Octoxynol-40 (1) | | |
| Methylchloroisothiazolinone (and) | 0.1 | Preservative |
| Methylisothiazolinone | | |

Results:

Example 10 provides a hair conditioner example formulation wherein the di-t-butoxytetramethyldisiloxane provides advantageous low-resistance wet-combing and shorter drying time characteristics. The di-t-butoxytetramethyldisiloxane- and cyclopentasiloxane- containing formulations exhibit no difference in the visual stability test performance.

| | 1 Week RT | 1 Week 50 C. |
|---|---|---|
| E10 | Ok | ok |
| CE10 | ok | ok |

### Example 11

### Conditioning Shampoo

This anionic formulation is typical for rinse-off applications and, with minor modifications, can be regarded as representative for shampoos, shampoos & conditioners, body washes, bath gels, hand soaps and the like.

Formulation E11:

| | Part/ | |
|---|---|---|
| | Wt | |
| Ingredient | (%) | Function |
| PART A | | |
| Ammonium Lauryl Sulfate | 25.00 | Primary Emulsifier |
| Ammonuim Laureth Sulfate | 10.00 | Co-Emulsifier |
| Cocamide-MEA | 4.00 | Surfactant, |
| | | foam booster |
| PEG-5 Cocamide | 4.00 | Surfactant |
| Di-t-butoxytetramethyldisiloxane (1) | 4.00 | Conditioner, Antifoam |
| Propylene Glycol | 3.00 | Moisturizer, Skin Feel |
| Isopropylaminedodecylbenzenesulfonate | 2.00 | Surfactant |
| (IDBS) | | |
| Guar Hydroxypropyltrimonium Chloride | 1.00 | Conditioning thickener |
| Laureth-23 | 0.23 | Surfactant |
| Citric Acid | 0.20 | pH adjuster |
| Ammonium Chloride | 2.00 | Thickener |
| Water | 44.52 | Solvent |

Procedure:

1. At 400 rpm, add low-charge-density guar to water, wait 15 min, then add CocamideMEA, PEG-5 Cocamide.

2. Heat to 60 C. Then add D5, PG and Laureth-23. Then add anionics in the following order: IDBS, ALES, ALS. Then add NH4Cl, then add Citric Acid yielding pH 6. Stir at 600 rpm for 30 min, then cool to RT.

Trade Names/Suppliers: (1) GE Silicones

### Comparative Example 11

Here, cyclopentasiloxane replaces di-t-butoxytetramethyldisiloxane (Formulation CE11) using the same process conditions as outlined above:

| | Part/ | |
|---|---|---|
| | Wt | |
| Ingredient | (%) | Function |
| PART A | | |
| Ammonium Lauryl Sulfate | 25.00 | Primary Emulsifier |
| Ammonuim Laureth Sulfate | 10.00 | Co-Emulsifier |
| Cocamide-MEA | 4.00 | Surfactant, |
| | | foam booster |
| PEG-5 Cocamide | 4.00 | Surfactant |
| Cyclopentasiloxane (1) | 4.00 | Conditioner, Antifoam |
| Propylene Glycol | 3.00 | Moisturizer, Skin Feel |
| Isopropylamine-dodecylbenzenesulfonate | 2.00 | Surfactant |
| Guar Hydroxypropyltrimonium Chloride | 1.00 | Conditioning thickener |
| Laureth-23 | 0.23 | Surfactant |
| Citric Acid | 0.20 | pH adjuster |
| Ammonium Chloride | 2.00 | Thickener |
| Water | 44.52 | Solvent |

Trade Names/Suppliers: (1) GE Silicones
Results:

Example 11 provides an example for a typical anionic rinse-off formulation, in this particular case for a conditioning shampoo. Here, the di-t-butoxytetramethyldisiloxane provides foam control and conditioning. The di-t-butoxytetramethyldisiloxane- and cyclopentasiloxane-containing formulations exhibit no difference in the visual stability test performance.

| | 1 Week | 1 Week | 2 Weeks | 2 Weeks | 4 Weeks | 4 Weeks |
|---|---|---|---|---|---|---|
| | RT | 50 C. | RT | 50 C. | RT | 50 C. |
| E10 | ok | ok | ok | ok | ok | ok |
| CE10 | ok | ok | ok | ok | ok | ok |

### Example 12

### Fabric Cleaning I

Example 12: A 100% untreated cotton swatch of size 1″×1″ was soiled with 0.02 g of motor oil (Pennzoil 10W-30). The swatch was then cleaned by rinsing in 5 g of di-t-butoxytetramethyl disiloxane for 30 seconds. The swatch was then dried at 90 C for 2 minutes. Visual examination showed that the stain was completely removed.

Comparative Example 12: A 100% untreated cotton swatch of size 1″×1″ was soiled with 0.02 g of motor oil (Pennzoil 10W-30). The swatch was then cleaned by rinsing in 5 g of cyclopentasiloxane for 30 seconds. The swatch was then dried at 90 C for 2 minutes. Visual examination showed that the stain was not completely removed leaving behind a light yellow mark.

### Example 13

### Fabric Cleaning II

Example 13: A 100% untreated cotton swatch of size 1″×1″ was soiled with 0.02 g of yellow vacuum pump oil 19 (VWR). The swatch was then cleaned by rinsing in 5 g of di-t-butoxytetramethyl disiloxane for 30 seconds. The swatch was then dried at 90 C for 2 minutes. Visual examination showed that the stain was completely removed.

### Comparative Example 13

A 100% untreated cotton swatch of size 1″×1″ was soiled with 0.02 g of yellow vacuum pump oil 19 (VWR). The swatch was then cleaned by rinsing in 5 g of cyclopentasiloxane for 30 seconds. The swatch was then dried at 90 C for 2 minutes. Visual examination showed that the stain was completely removed.

### Example 14

### Metal Cleaning I

Example 14: A stainless steel metal spring weighing 2.0 g was soiled by immersing in yellow vacuum pump oil 19 (VWR). The spring was then cleaned by rinsing in 5 g of di-t-butoxytetramethyl disiloxane for 30 seconds. The spring was then dried at 90 C for 2 minutes. Examination by feel showed that the oil was completely removed from the spring.

### Comparative Example 14

A stainless steel metal spring weighing 2.0 g was soiled by immersing in yellow vacuum pump oil 19 (VWR). The spring was then cleaned by rinsing in 5 g of cyclopentasiloxane for 30 seconds. The spring was then dried at 90 C for 2 minutes. Examination by feel showed that the oil was completely removed from the spring.

Silicon/Hydrocarbon Balance

| Molecule | Wt % Si | Wt % Hydrocarbons |
|---|---|---|
| Cyclopentasiloxane | 37.87 | 40.55 |
| Di-t-butoxytetramethyldisiloxane | 20.17 | 62.61 |
| Tri-t-butoxymethylsilane | 10.70 | 70.01 |
| Di-t-butoxydimethylsilane | 13.74 | 70.61 |
| Di-isopropoxytetramethyldisiloxane | 22.42 | 58.41 |

| | | | Di-t- |
|---|---|---|---|
| Solvent | Wt. % | Cyclopentasiloxane | butoxytetramethyldisiloxane |
| Butylmethoxydibenzoylmethane | 1 | Insoluble at RT | Partially soluble at RT |
| Butylmethoxydibenzoylmethane | 1 | Partially soluble at | Completely soluble at 75° C., |
| | | 75° C., precipitates | soluble even on cooling |
| | | out of solution on | |
| | | cooling | |
| 4-methyl benzylidene camphor | 1 | Partially soluble at | Soluble at RT |
| | | RT | |
| Octocrylene | 10 | Insoluble | Completely soluble |
| Benzophenone-4 | 1 | Insoluble at 75° C. | Soluble at 75° C. |
| Benzophenone-3 | 2.5 | Insoluble at RT | Soluble at RT |
| Benzophenone-3 | 10 | Insoluble at 75° C. | Soluble at 75° C. |
| Petrolatum | 10 | Partially soluble @ | Completely soluble at 75° C. |
| | | 75° C. | |
| Propylene glycol | 1 | Partially soluble at | Fully soluble at RT |
| | | RT | |
| Cetyl alcohol | 10 | Soluble at 75° C., | Soluble at 75° C., precipitation |
| | | preicipitates out of | is very slow |
| | | solution at RT | |
| | | rapidly | |

1. A retail silicone composition comprising:
(a) an organic compound having a solubility in a cyclic siloxane and
(b) a silicone composition comprising an evaporable siloxane having the molecular formula:
MᵥM′_{w}DₓT_{y}Q_{z}
where the subscripts have the following values: x=0, 1, 2, 3, or 4; y=0, 1, or 2; z=0, 1, 2, 3, or 4; and v and w may be zero or a positive integer subject to the limitation that v+w≧1 where the components are defined as follows:
M=(R¹O)ₐ(R²O)_{b}R³_{c}SiO_{1/2} where the subscripts a, b, and c have the following values: a=1, 2, or 3; b=0, 1, or 2; c=0, 1, or 2; subject to the limitation that a+b+c=3 except when w+x+y+z=0 then a+b+c=4;
M′=(R¹O)_{d}(R²O)ₑR⁴_{f}SiO_{1/2} where the subscripts d, e, and f have the following values: d=1, 2, or 3; e=0, 1, or 2; f=0, 1, or 2; subject to the limitation that d+e+f=3;
D=R⁵R⁶SiO_{2/2};
T=R⁷SiO_{3/2}; and
Q=SiO_{4/2};
where R¹ and R² are selected from the group of linear or branched monovalent hydrocarbon radicals having from one to eight carbon atoms and where each R³, R⁴, R⁵, R⁶, and R⁷ is independently selected from the group of monovalent hydrocarbon radicals having from one to sixty carbon atoms, wherein said organic compound has a solubility in said evaporable siloxane equal to or greater than the solubility of said organic compound in a cyclic siloxane of equal or greater molecular weight relative to the molecular weight of said evaporable siloxane.

2. The evaporable siloxane composition of claim 1 where the subscript z is zero.

3. The evaporable siloxane of claim 2 where the subscript y is zero.

4. The evaporable siloxane of claim 3 where R³ is methyl.

5. The evaporable siloxane of claim 4 where R¹ and R² are independently selected from the group consisting of propyl, i-propyl, butyl, i-butyl and t-butyl.

6. A retail silicone composition comprising:
(a) an organic compound having a solubility in a cyclic siloxane and
(b) an aqueous emulsion where the discontinuous phase comprises water and the continuous phase comprises an evaporable siloxane having the molecular formula:
MᵥM′_{w}DₓT_{y}Q_{z}
where the subscripts have the following values: x=0, 1, 2, 3, or 4; y=0, 1, or 2; z=0, 1, 2, 3, or 4; and v and w may be zero or a positive integer subject to the limitation that v+w≧1 where the components are defined as follows:
M=(R¹O)ₐ(R²O)_{b}R³_{c}SiO_{1/2} where the subscripts a, b, and c have the following values: a=1, 2, or 3; b=0, 1, or 2; c=0, 1, or 2; subject to the limitation that a+b+c=3 except when w+x+y+z=0 then a+b+c=4;
M′=(R¹O)_{d}(R²O)ₑR⁴_{f}SiO_{1/2} where the subscripts d, e, and f have the following values: d=1, 2, or 3; e=0, 1, or 2; f=0, 1, or 2; subject to the limitation that d+e+f=3;
D=R⁵R⁶SiO_{2/2};
T=R⁷SiO_{3/2}; and
Q=SiO_{4/2};
where R¹ and R² are selected from the group of linear or branched monovalent hydrocarbon radicals having from one to eight carbon atoms and where each R³, R⁴, R⁵, R⁶, and R⁷ is independently selected from the group of monovalent hydrocarbon radicals having from one to sixty carbon atoms, wherein said organic compound has a solubility in said evaporable siloxane equal to or greater than the solubility of said organic compound in a cyclic siloxane of equal or greater molecular weight relative to the molecular weight of said evaporable siloxane.

7. The evaporable siloxane of claim 6 where the subscript z is zero.

8. The evaporable siloxane of claim 7 where the subscript y is zero.

9. The evaporable siloxane of claim 8 where R³ is methyl.

10. The evaporable siloxane of claim 9 where R¹ and R² are independently selected from the group consisting of propyl, i-propyl, butyl, i-butyl and t-butyl.

11. A retail silicone composition comprising:
(a) an organic compound having a solubility in a cyclic siloxane and
(b) an aqueous emulsion where the continuous phase comprises water and the discontinuous phase comprises an evaporable siloxane having the molecular formula:
MᵥM′_{w}DₓT_{y}Q_{z}
where the subscripts have the following values: x=0, 1, 2, 3, or 4; y=0, 1, or 2; z=0, 1, 2, 3, or 4; and v and w may be zero or a positive integer subject to the limitation that v+w≧1 where the components are defined as follows:
M=(R¹O)ₐ(R²O)_{b}R³_{c}SiO_{1/2} where the subscripts a, b, and c have the following values: a=1, 2, or 3; b=0, 1, or 2; c=0, 1, or 2; subject to the limitation that a+b+c=3 except when w+x+y+z=0 then a+b+c=4;
M′=(R¹O)_{d}(R²O)ₑR⁴_{f}O_{1/2} where the subscripts d, e, and f have the following values: d=1, 2, or 3; e=0, 1, or 2; f=0, 1, or 2; subject to the limitation that d+e+f=3;
D=R⁵R⁶SiO_{2/2};
T=R⁷SiO_{3/2}; and
Q=SiO_{4/2};
where R¹ and R² are selected from the group of linear or branched monovalent hydrocarbon radicals having from one to eight carbon atoms and where each R³, R⁴, R⁵, R⁶, and R⁷ is independently selected from the group of monovalent hydrocarbon radicals having from one to sixty carbon atoms, wherein said organic compound has a solubility in said evaporable siloxane equal to or greater than the solubility of said organic compound in a cyclic siloxane of equal or greater molecular weight relative to the molecular weight of said evaporable siloxane.

12. The evaporable siloxane of claim 11 where the subscript z is zero.

13. The evaporable siloxane of claim 12 where the subscript y is zero.

14. The evaporable siloxane of claim 13 where R³ is methyl.

15. The evaporable siloxane of claim 14 where R¹ and R² are independently selected from the group consisting of propyl, i-propyl, butyl, i-butyl and t-butyl.

16. A retail silicone composition comprising:
(a) an organic compound having a solubility in a cyclic siloxane and
(b) a non-aqueous emulsion where the discontinuous phase comprises a non-aqueous hydroxylic solvent and the continuous phase comprises an evaporable siloxane having the molecular formula:
MᵥM′_{w}DₓT_{y}Q_{z}
where the subscripts have the following values: x=0, 1, 2, 3, or 4; y=0, 1, or 2; z=0, 1, 2, 3, or 4; and v and w may be zero or a positive integer subject to the limitation that v+w≧1 where the components are defined as follows:
M=(R¹O)ₐ(R²O)_{b}R³_{c}SiO_{1/2} where the subscripts a, b, and c have the following values: a=1, 2, or 3; b=0, 1, or 2; c=0, 1, or 2; subject to the limitation that a+b+c=3 except when w+x+y+z=0 then a+b+c=4;
M′=(R¹O)_{d}(R²O)ₑR⁴_{f}SiO_{1/2} where the subscripts d, e, and f have the following values: d=1, 2, or 3; e=0, 1, or 2; f=0, 1, or 2; subject to the limitation that d+e+f=3;
D=R⁵R⁶SiO_{2/2};
T=R⁷SiO_{3/2}; and
Q=SiO_{4/2};
where R¹ and R² are selected from the group of linear or branched monovalent hydrocarbon radicals having from one to eight carbon atoms and where each R³, R⁴, R⁵, R⁶, and R⁷ is independently selected from the group of monovalent hydrocarbon radicals having from one to sixty carbon atoms, wherein said organic compound has a solubility in said evaporable siloxane equal to or greater than the solubility of said organic compound in a cyclic siloxane of equal or greater molecular weight relative to the molecular weight of said evaporable siloxane.

17. The evaporable siloxane of claim 16 where the subscript z is zero.

18. The evaporable siloxane of claim 17 where the subscript y is zero.

19. The evaporable siloxane of claim 18 where R³ is methyl.

20. The evaporable siloxane of claim 19 where R¹ and R² are independently selected from the group consisting of propyl, i-propyl, butyl, i-butyl and t-butyl.

21. A retail silicone composition comprising:
(a) an organic compound having a solubility in a cyclic siloxane and
(b) a non-aqueous emulsion where the continuous phase comprises a non-aqueous hydroxylic solvent and the discontinuous phase comprises a an evaporable siloxane having the molecular formula:
MᵥM′_{w}DₓT_{y}Q_{z}
where the subscripts have the following values: x=0, 1, 2, 3, or 4; y=0, 1, or 2; z=0, 1, 2, 3, or 4; and v and w may be zero or a positive integer subject to the limitation that v+w≧1 where the components are defined as follows:
M=(R¹O)ₐ(R²O)_{b}R³_{c}SiO_{1/2} where the subscripts a, b, and c have the following values: a=1, 2, or 3; b=0, 1, or 2; c=0, 1, or 2; subject to the limitation that a+b+c=3 except when w+x+y+z=0 then a+b+c=4;
M′=(R¹O)_{d}(R²O)ₑR⁴_{f}SiO_{1/2} where the subscripts d, e, and f have the following values: d=1, 2, or 3; e=0, 1, or 2; f=0, 1, or 2; subject to the limitation that d+e+f=3;
D=R⁵R⁶SiO_{2/2};
T=R⁷SiO_{3/2}; and
Q=SiO_{4/2};
where R¹ and R² are selected from the group of linear or branched monovalent hydrocarbon radicals having from one to eight carbon atoms and where each R³, R⁴, R⁵, R⁶, and R⁷ is independently selected from the group of monovalent hydrocarbon radicals having from one to sixty carbon atoms, wherein said organic compound has a solubility in said evaporable siloxane equal to or greater than the solubility of said organic compound in a cyclic siloxane of equal or greater molecular weight relative to the molecular weight of said evaporable siloxane.

22. The evaporable siloxane of claim 21 where the subscript z is zero.

23. The evaporable siloxane of claim 22 where the subscript y is zero.

24. The evaporable siloxane of claim 23 where R³ is methyl.

25. The evaporable siloxane of claim 24 where R¹ and R² are independently selected from the group consisting of propyl, i-propyl, butyl, i-butyl and t-butyl.

26. A retail silicone composition comprising:
(a) an organic compound and
(b) a silicone composition comprising an evaporable siloxane having the molecular formula:
MᵥM′_{w}DₓT_{y}Q_{z}
where the subscripts have the following values: x=0, 1, 2, 3, or 4; y=0, 1, or 2; z=0, 1, 2, 3, or 4; and v and w may be zero or a positive integer subject to the limitation that v+w≧1 where the components are defined as follows:
M=(R¹O)ₐ(R²O)_{b}R³_{c}SiO_{1/2} where the subscripts a, b, and c have the following values: a=1, 2, or 3; b=0, 1, or 2; c=0, 1, or 2; subject to the limitation that a+b+c=3 except when w+x+y+z=0 then a+b+c=4;
M′=(R¹O)_{d}(R²O)ₑR⁴_{f}SiO_{1/2} where the subscripts d, e, and f have the following values: d=1, 2, or 3; e=0, 1, or 2; f=0, 1, or 2; subject to the limitation that d+e+f=3;
D=R⁵R⁶SiO_{2/2};
T=R⁷SiO_{3/2}; and
Q=SiO_{4/2};
where R¹ and R² are selected from the group of linear or branched monovalent hydrocarbon radicals having from one to eight carbon atoms and where each R³, R⁴, R⁵, R⁶, and R⁷ is independently selected from the group of monovalent hydrocarbon radicals having from one to sixty carbon atoms, wherein said organic compound and said evaporable siloxane in said retail silicone composition exhibit compatibility.

27. The evaporable siloxane composition of claim 26 where the subscript z is zero.

28. The evaporable siloxane of claim 27 where the subscript y is zero.

29. The evaporable siloxane of claim 28 where R³ is methyl.

30. The evaporable siloxane of claim 29 where R¹ and R² are independently selected from the group consisting of propyl, i-propyl, butyl, i-butyl and t-butyl.

31. A retail silicone composition comprising:
(a) an organic compound and
(b) an aqueous emulsion where the discontinuous phase comprises water and the continuous phase comprises an evaporable siloxane having the molecular formula:
MᵥM′_{w}DₓT_{y}Q_{z}
where the subscripts have the following values: x=0, 1, 2, 3, or 4; y=0, 1, or 2; z=0, 1, 2, 3, or 4; and v and w may be zero or a positive integer subject to the limitation that v+w≧1 where the components are defined as follows:
M=(R¹O)ₐ(R²O)_{b}R³_{c}SiO_{1/2} where the subscripts a, b, and c have the following values: a=1, 2, or 3; b=0, 1, or 2; c=0, 1, or 2; subject to the limitation that a+b+c=3 except when w+x+y+z=0 then a+b+c=4;
M′=(R¹O)_{d}(R²O)ₑR⁴_{f}SiO_{1/2} where the subscripts d, e, and f have the following values: d=1, 2, or 3; e=0, 1, or 2; f=0, 1, or 2; subject to the limitation that d+e+f=3;
D=R⁵R⁶SiO_{2/2};
T=R⁷SiO_{3/2}; and
Q=SiO_{4/2};
where R¹ and R² are selected from the group of linear or branched monovalent hydrocarbon radicals having from one to eight carbon atoms and where each R³, R⁴, R⁵, R⁶, and R⁷ is independently selected from the group of monovalent hydrocarbon radicals having from one to sixty carbon atoms, wherein said organic compound and said evaporable siloxane in said retail silicone composition exhibit compatibility.

32. The evaporable siloxane of claim 31 where the subscript z is zero.

33. The evaporable siloxane of claim 32 where the subscript y is zero.

34. The evaporable siloxane of claim 33 where R³ is methyl.

35. The evaporable siloxane of claim 34 where R¹ and R² are independently selected from the group consisting of propyl, i-propyl, butyl, i-butyl and t-butyl.

36. A retail silicone composition comprising:
(a) an organic compound and
(b) an aqueous emulsion where the continuous phase comprises water and the discontinuous phase comprises an evaporable siloxane having the molecular formula:
MᵥM′_{w}DₓT_{y}Q_{z}
where the subscripts have the following values: x=0, 1, 2, 3, or 4; y=0, 1, or 2; z=0, 1, 2, 3, or 4; and v and w may be zero or a positive integer subject to the limitation that v+w≧1 where the components are defined as follows:
M=(R¹O)ₐ(R²O)_{b}R³_{c}SiO_{1/2} where the subscripts a, b, and c have the following values: a=1, 2, or 3; b=0, 1, or 2; c=0, 1, or 2; subject to the limitation that a+b+c=3 except when w+x+y+z=0 then a+b+c=4;
M′=(R¹O)_{d}(R²O)ₑR⁴_{f}SiO_{1/2} where the subscripts d, e, and f have the following values: d=1, 2, or 3; e=0, 1, or 2; f=0, 1, or 2; subject to the limitation that d+e+f=3;
D=R⁵R⁶SiO_{2/2};
T=R⁷SiO_{3/2}; and
Q=SiO_{4/2};
where R¹ and R² are selected from the group of linear or branched monovalent hydrocarbon radicals having from one to eight carbon atoms and where each R³, R⁴, R⁵, R⁶, and R⁷ is independently selected from the group of monovalent hydrocarbon radicals having from one to sixty carbon atoms, wherein said organic compound and said evaporable siloxane in said retail silicone composition exhibit compatibility.

37. The evaporable siloxane of claim 36 where the subscript z is zero.

38. The evaporable siloxane of claim 37 where the subscript y is zero.

39. The evaporable siloxane of claim 38 where R³ is methyl.

40. The evaporable siloxane of claim 39 where R¹ and R² are independently selected from the group consisting of propyl, i-propyl, butyl, i-butyl and t-butyl.

41. A retail silicone composition comprising:
(a) an organic compound and
(b) a non-aqueous emulsion where the discontinuous phase comprises a non-aqueous hydroxylic solvent and the continuous phase comprises an evaporable siloxane having the molecular formula:
MᵥM′_{w}DₓT_{y}Q_{z}
where the subscripts have the following values: x=0, 1, 2, 3, or 4; y=0, 1, or 2; z=0, 1, 2, 3, or 4; and v and w may be zero or a positive integer subject to the limitation that v+w≧1 where the components are defined as follows:
M=(R¹O)ₐ(R²O)_{b}R³_{c}SiO_{1/2} where the subscripts a, b, and c have the following values: a=1, 2, or 3; b=0, 1, or 2; c=0, 1, or 2; subject to the limitation that a+b+c=3 except when w+x+y+z=0 then a+b+c=4;
M′=(R¹O)_{d}(R²O)ₑR⁴_{f}SiO_{1/2} where the subscripts d, e, and f have the following values: d=1, 2, or 3; e=0, 1, or 2; f=0, 1, or 2; subject to the limitation that d+e+f=3;
D=R⁵R⁶SiO_{2/2};
T=R⁷SiO_{3/2}; and
Q=SiO_{4/2};
where R¹ and R² are selected from the group of linear or branched monovalent hydrocarbon radicals having from one to eight carbon atoms and where each R³, R⁴, R⁵, R⁶, and R⁷ is independently selected from the group of monovalent hydrocarbon radicals having from one to sixty carbon atoms, wherein said organic compound and said evaporable siloxane in said retail silicone composition exhibit compatibility.

42. The evaporable siloxane of claim 41 where the subscript z is zero.

43. The evaporable siloxane of claim 42 where the subscript y is zero.

44. The evaporable siloxane of claim 43 where R³ is methyl.

45. The evaporable siloxane of claim 44 where R¹ and R² are independently selected from the group consisting of propyl, i-propyl, butyl, i-butyl and t-butyl.

46. A retail silicone composition comprising:
(a) an organic compound and
(b) a non-aqueous emulsion where the continuous phase comprises a non-aqueous hydroxylic solvent and the discontinuous phase comprises a an evaporable siloxane having the molecular formula:
MᵥM′_{w}DₓT_{y}Q_{z}
where the subscripts have the following values: x=0, 1, 2, 3, or 4; y=0, 1, or 2; z=0, 1, 2, 3, or 4; and v and w may be zero or a positive integer subject to the limitation that v+w≧1 where the components are defined as follows:
M=(R¹O)ₐ(R²O)_{b}R³_{c}SiO_{1/2} where the subscripts a, b, and c have the following values: a=1, 2, or 3; b=0, 1, or 2; c=0, 1, or 2; subject to the limitation that a+b+c=3 except when w+x+y+z=0 then a+b+c=4;
M′=(R¹O)_{d}(R²O)ₑR⁴_{f}SiO_{1/2} where the subscripts d, e, and f have the following values: d=1, 2, or 3; e=0, 1, or 2; f=0, 1, or 2; subject to the limitation that d+e+f=3;
D=R⁵R⁶SiO_{2/2};
T=R⁷SiO_{3/2}; and
Q=SiO_{4/2};
where R¹ and R² are selected from the group of linear or branched monovalent hydrocarbon radicals having from one to eight carbon atoms and where each R³, R⁴, R⁵, R⁶, and R⁷ is independently selected from the group of monovalent hydrocarbon radicals having from one to sixty carbon atoms, wherein said organic compound and said evaporable siloxane in said retail silicone composition exhibit compatibility.

47. The evaporable siloxane of claim 46 where the subscript z is zero.

48. The evaporable siloxane of claim 47 where the subscript y is zero.

49. The evaporable siloxane of claim 48 where R³ is methyl.

50. The evaporable siloxane of claim 49 where R¹ and R² are independently selected from the group consisting of propyl, i-propyl, butyl, i-butyl and t-butyl.
